(19) United States
(12) Patent Application Publication
(10) Pub. No.: US 2006/0073206 A1
(43) Pub. Date: Apr. 6, 2006

(54) Coating meltable substances and substance mixtures

(76) Inventors: Sandra Hoffmann, Munich (DE); Peter Victor, Duesseldorf (DE)

Correspondence Address: DANN DORFMAN HERRELL AND SKILLMAN;A PROFESSIONAL CORPORATION, 1601 MARKET STREET, SUITE 2400, PHILADELPHIA, PA, 19103-2307, US

(21) Appl. No.: 11/224,667

(22) Filed: Sep. 12, 2005

Related U.S. Application Data
(63) Continuation of application No. PCT/EP04/02107, filed on Mar. 3, 2004.

(30) Foreign Application Priority Data
Mar. 12, 2003 (DE) DE 103 10 679.0

(51) Int. Cl. A61K 9/28; A61K 9/24; B01J 13/00

(52) U.S. Cl. 424/471; 427/2.14

(57) ABSTRACT

The invention relates to a method for coating meltable substances or substance mixtures. A melt of the substance (mixture) drips into a melt of the coating agent(s) where it solidifies at least on the surface thereof and the solidified melt, which is coated on at least the surface thereof, is subsequently separated from the melt of the coating agent(s)

### CROSS-REFERENCE TO RELATED APPLICATIONS

This application is a continuation under 35 U.S.C. § 365(a) and 35 U.S.C. § 120 of International Application PCT/EP2004/002107, filed Mar. 3, 2004. This application also claims priority under 35 U.S.C. § 119 of DE 103 10 679.0, filed Mar. 12, 2003, which is incorporated herein by reference in its entirety.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF THE INVENTION

(1) Field of the Invention

The present invention concerns a method for encasing substances, namely meltable substances. It concerns in particular the encasing of substances that can be used in washing or cleaning agents.

The coating of substances for technical and/or aesthetic reasons is widely known in the existing art. For example, pharmaceuticals are often equipped with coatings to make them applicable more easily and/or releasable only at certain times. Coating with colored coating materials for aesthetic reasons or for reasons of distinguishability is also widely used in this context. In technical products, coatings often serve to protect the coated substances from environmental influences. For example, enzymes are often coated in order to protect them from atmospheric oxygen or aggressive substances. Similar instances exist in almost all branches of industry, for example the food, animal feed, construction materials, adhesives, cosmetics, and washing or cleaning agent industries.

As a result of the extensive use of coated substances, a plurality of methods exist for applying corresponding coatings onto substrates. One widespread method is the application of a melt, solution, dispersion, suspension, or emulsion of the coating material onto the substrates, and subsequent solidification or evaporation of solvents. By means of a melt, relatively thick coatings are obtained that of course consume a great deal of coating material. Thinner coatings can be achieved with the use of solutions (or dispersions, etc.), but here in some cases a considerable amount of solvent must be evaporated and, if applicable, recovered, which on the one hand decreases manufacturing throughputs and on the other hand raises costs. Coating methods are in general difficult to carry out on a continuous basis, since as the size of the batch to be coated increases, so do problems such as nonuniform coatings and/or adhesion of the individual substrate particles.

### BRIEF SUMMARY OF THE INVENTION

It was the object of the present invention to make available a simple, cost-effective coating method that can be carried out on a continuous basis.

It has now been found that meltable substances can be coated particularly well if they are melted and are introduced into a melt of the coating material.

The subject matter of the invention is, in a first embodiment, a method for encasing meltable substances or substance mixtures, in which a melt of the substance (mixture) is dripped into a melt of the encasing medium/media, allowed to solidify therein at least on its surface, and the encased melt, solidified at least on its surface, is subsequently separated from the melt of the encasing medium/media.

According to the present invention, firstly a substance or substance mixture to be coated is melted. This melt is dripped into a melt made up of one or more encasing materials, and allowed to solidify therein at least partially. Because of the temperature gradient between the melt of the material to be encased and the melt of the encasing medium, the former solidifies first on its surface, forming a droplet that has a liquid core and a casing that is made up on its inner side of solidified material to be encased, and on its outer side of encasing material. This element already possesses sufficient stability to be removed from the melt of the encasing material. Complete solidification of the liquid interior can take place during and/or after the removal operation. It is preferred in the context of the present invention, however, to perform the subsequent method steps only once the interior of the element described above has completely solidified. The solidification time necessary for this can be varied by selecting the melting points of the encasing material and of the material to be encased, and by way of the respective temperatures of those materials and the size of the particles introduced.

### BRIEF DESCRIPTION OF THE SEVERAL VIEWS OF THE DRAWING(S)

Not Applicable.

### DETAILED DESCRIPTION OF THE INVENTION

In the context of the present invention, the term “encasing material” is used both for an individual coating material and for a mixture of different coating materials. “Encasing material” therefore identifies the substance(s) that form(s) the later coating. Entirely analogously, the term “substance to be encased” is also not limited to one single substance; substance mixtures can also be referred to in this context. These mixtures can be melted together if no phase separations occur. It is also easily possible, however, to melt different substances separately and introduced them separately into a melt. In the former case a coated element containing multiple substances is obtained; in the latter case, each coated element contains only one substance.

The term “dripped into the melt” encompasses the introduction of the substance to be encased, in molten form. Technical methods that contain a similar step are, for example, microencapsulation, pastillization, pelletization, etc. The size of the encased particles can be varied by selecting the droplet size (see below).

The greater the difference between the melting points of the encasing material and of the substance to be encased, the better and more quickly the method according to the present invention can be carried out. The higher the melting temperature of the material to be encased, and the lower the melting temperature of the encasing material, the faster the material to be encased will solidify in the melt of the encasing material, at least on its surface. In addition, of course, the heat capacities of the substances also play a role.

Particularly preferred methods according to the present invention are characterized in that the melting point or melting range of the substance or substance mixture to be encased is at least 5° C., preferably at least 10° C., particularly preferably at least 20° C., more preferably at least 25° C., and in particular at least 30° C. above the melting point or melting range of the encasing medium.

Limits in terms of the melting points or melting point difference exist insofar as the encasing material is intended to form a stable casing on the material to be encased. Casings that already soften at room temperature are not reasonably usable in technical terms, for which reason—depending on the field of application of the method and of the products produced with the use thereof—the melting point of the encasing material should not be less than 20 to 25° C. Because of rising process costs, the melting point of the material to be encased should not exceed 500 to 600° C., although even much higher temperatures are easily achievable.

In methods that are particularly preferred according to the present invention, the melting point of the substance to be encased lies within the range from 30 to 300° C., preferably from 40 to 250° C., particularly preferably from 50 to 200° C., and in particular from 60 to 170° C.

Substances or substance mixtures from a very wide variety of substance classes are suitable as substances to be encased in the method according to the present invention. In terms of a preferred area of use of the end products of the method according to the present invention, meltable washing or cleaning agent ingredients are, in particular, preferred substances to be encased. Preferred methods according to the present invention are therefore characterized in that one or more ingredients of washing or cleaning agents are used as the substance to be encased.

Preferred substances to be encased derive, for example, from the group of the monomeric and/or polymeric organic acids, preferably from the group of the mono- and/or dicarboxylic acids, particularly preferably the surfactant acids, and/or from the group of the acids recited below.

One substance class that is outstandingly suitable as a substance to be encased is aliphatic and aromatic dicarboxylic acids, which can be melted and processed according to the present invention individually, in a mixture with one another, or even in a mixture with other substances. Particularly preferred dicarboxylic acids are summarized in the table below:

| Trivial name | IUPAC name | Melting point (° C.) |
|---|---|---|
| Oxalic acid | Ethanedioic acid | 101.5 |
| Malonic acid | Propanedioic acid | 135 |
| Succinic acid | Butanedioic acid | 185 |
| Glutaric acid | Pentanedioic acid | 97 |
| Adipic acid | Hexaneidoic acid | 153 |
| Pimelic acid | Heptanedioic acid | 105 |
| Azelaic acid | Nonaneidoic acid | 106 |
| Sebacic acid | Decanedioic acid | 134.5 |
| | Dodecanedioic acid | 128 |
| Maleic acid | (Z)-butenedioic acid | 130-139 |
| Fumaric acid | (E)-butenedioic acid | 287 |
| Sorbic acid | 2,4-hexadienedioic acid | 134 |
| Phthalic acid | 1,2-benzenedicarboxylic acid | 208 |
| Terephthalic acid | 1,4-benzenedicarboxylic acid | |

Instead of the aforesaid dicarboxylic acids or in a mixture with them, the corresponding anhydrides can also be used; this is advantageous in particular in the case of citric acid, glutaric acid, maleic acid, and phthalic acid.

In addition to the dicarboxylic acids, carboxylic acids and their salts are also suitable as materials to be encased. Of this substance class, in particular citric acid and trisodium citrate, as well as salicylic acid and glycolic acid, have proven suitable. It is also possible, in particularly advantageous fashion, to use fatty acids, preferably having more than 10 carbon atoms, and their salts, as materials to be encased. Carboxylic acids usable in the context of the present invention are, for example, hexanoic acid (caproic acid), heptanoic acid (oenanthic acid), octanoic acid (caprylic acid), nonanoic acid (pelargonic acid), decanoic acid (capric acid), undecanoic acid, etc. It is preferred in the context of the present invention to use fatty acids such as dodecanoic acid (lauric acid), tetradecanoic acid (myristic acid), hexadecanoic acid (palmitic acid), octadecanoic acid (stearic acid), eicosanoic acid (arachidic acid), docosanoic acid (behenic acid), tetracosanoic acid (lignoceric acid), hexacosanoic acid (cerotinic acid), triacontanoic acid (melissic acid), as well as the unsaturated species 9c-hexadecenoic acid (palmitoleic acid), 6c-octadeceneoic acid (petroselinic acid), 6t-octadecenoic acid (petroselaidic acid), 9c-octadecenoic acid (oleic acid), 9t-octadecenoic acid (elaidic acid), 9c, 12c-octadecadienoic acid (linoleic acid), 9t, 12t-octadecadienoic acid (linolaidic acid), and 9c, 12c, 15c-octadecatrienoic acid (linolenic acid). For cost reasons, it is preferred to use not the pure species but instead technical mixtures of the individual acids that are accessible by means of fat cleavage. Such mixtures are, for example, coconut oil fatty acid (approx. 6 wt % C₈, 6 wt % C₁₀, 48 wt % C₁₂, 18 wt % C₁₄, 10 wt % C₁₆, 2 wt % C₁₈, 8 wt % C_{18′}, 1 wt % C_{18″}), palm oil fatty acid (approx. 4 wt % C₈, 5 wt % C₁₀, 50 wt % C₁₂, 15 wt % C₁₄, 7 wt % C₁₆, 2 wt % C₁₈, 15 wt % C_{18′}1 wt % C_{18″}), tallow fatty acid (approx. 3 wt % C₁₄, 26 wt % C₁₆, 2 wt % C_{16′}, 2 wt % C₁₇, 17 wt % C₁₈, 44 wt % C_{18′}, 3 wt % C_{18″}, 1 wt % C_{18′″}), hardened tallow fatty acid (approx. 2 wt % C₁₄, 28 wt % C₁₆, 2 wt % C₁₇, 63 wt % C₁₈, 1 wt % C_{18′}), technical oleic acid (approx. 1 wt % C₁₂, 3 wt % C₁₄, 5 wt % C₆, 6 wt % C_{16′}, 1 wt % C₁₇, 2 wt % C₁₈, 70 wt % C_{18′}, 10 wt % C_{18″}, 0.5 wt % C_{18′″}), technical palmitic/stearic acid (approx. 1 wt % C₁₂, 2 wt % C₁₄, 45 wt % C₁₆, 2 wt % C₁₇, 47 wt % C₁₈, 1 wt % C_{18′}), and soybean oil fatty acid (approx. 2 wt % C₁₄, 15 wt % C₁₇, 47 wt % C₁₈, 1wt % C_{18′}, 45 wt % C_{18″}, 7 wt % C_{18′″}).

The aforementioned carboxylic acids are for the most part obtained industrially from natural fats and oils by hydrolysis. Whereas alkaline saponification, already performed in the last century, resulted directly in the alkaline salts (soaps), what is used today on an industrial scale for cleavage is only water, which cleaves the fats into glycerol and the free fatty acids. Methods applied industrially are, for example, cleavage in autoclaves or continuous high-pressure cleavage. The alkali metal salts of the aforementioned carboxylic acids or carboxylic acid mixtures can also be used, optionally mixed with other materials, for the method according to the present invention. In addition to these soaps, other anionic surfactant acids are also suitable for the method according to the present invention. Particularly important representatives of this substance class are described below.

Sulfuric acid semi-esters of long-chain alcohols are also anionic surfactants in their acid form, and usable in the context of the present invention. Their alkali metal salts, in particular sodium salts, the fatty alcohol sulfates, are accessible industrially from fatty alcohols, which are reacted with sulfuric acid, chlorosulfonic acid, amidosulfonic acid, or sulfur trioxide to yield the relevant alkyl sulfuric acids, and then neutralized. The fatty alcohols are obtained from the relevant fatty acids or fatty acid mixtures by high-pressure hydrogenation of the fatty acid methyl esters. The most important industrial process, by volume, for the production of fatty alkyl sulfuric acids is sulfonation of the alcohols with SO₃/air mixtures in special cascade, falling-film, or tube-bundle reactors.

A further class of anionic surfactant acids that can be used in the method according to the present invention is the alkyl ether sulfuric acids; their salts (the alkyl ether sulfates) are distinguished by comparison with the alkyl sulfates by a higher water solubility and lower sensitivity to water hardness (solubility of Ca salts). Alkyl ether sulfuric acids, like the alkyl sulfuric acids, are synthesized from fatty alcohols, which are reacted with ethylene oxide to yield the relevant fatty alcohol ethoxylates. Propylene oxide can also be used instead of ethylene oxide. Subsequent sulfonation with gaseous sulfur trioxide in short-term sulfonation reactors produces more than 98% yields of the relevant alkyl ether sulfuric acids.

Alkanesulfonic acids and olefinsulfonic acids are also usable in the context of the present invention as anionic surfactants in acid form. Alkanesulfonic acids can contain the sulfonic acid group in terminally bonded fashion (primary alkanesulfonic acids) or along the carbon chain (secondary alkanesulfonic acids); only the secondary alkanesulfonic acids are of commercial significance. These are produced by sulfochlorination or sulfoxidation of linear hydrocarbons. In sulfochlorination according to Reed, n-alkanes are reacted with sulfur dioxide and chlorine under UV light irradiation to produce the corresponding sulfochlorides, which directly yield the alkanesulfonates upon hydrolysis with alkalis, and the alkanesulfonic acids upon reaction with water. Because di- and polysulfochlorides as well as chlorinated hydrocarbons can occur during sulfochlorination as byproducts of the radical reaction, the reaction is usually performed only up to conversion rates of 30% and then halted.

Another process for producing alkanesulfonic acids is sulfoxidation, in which n-alkanes are reacted with sulfur dioxide and oxygen under UV light irradiation. This radical reaction produces successive alkylsulfonyl radicals that react further with oxygen to yield the alkylpersulfonyl radicals. The reaction with unconverted alkane yields an alkyl radical and the alkylpersulfonic acid, which decomposes into an alkyl peroxysulfonyl radical and a hydroxyl radical. The reaction of the two radicals with unconverted alkane produces the alkylsulfonic acids and water, which reacts with alkylpersulfonic acid and sulfur dioxide to produce sulfuric acid. To maximize the yield of the two end products (alkylsulfonic acid and sulfuric acid) and to suppress secondary reactions, this reaction is usually performed only up to conversion rates of 1% and then halted.

Olefinsulfonates are produced industrially by reacting α-olefins with sulfur trioxide. This forms intermediary zwitterions that cyclize into so-called sultones. Under suitable conditions (alkaline or acid hydrolysis), these sultones reaction to form hydroxyl alkanesulfonic acids or alkenesulfonic acids, both of which can likewise be used as anionic surfactant acids.

Alkyl benzene sulfonates have been known as high-performance anionic surfactants since the 1930s. At that time alkyl benzenes were produced by monochlorination of Kogasin fractions and subsequent Friedel-Crafts alkylation, and then sulfonated with oleum and neutralized with sodium hydroxide. In the early 1950s, alkyl benzene sulfonates were produced by tetramerizing propylene to form branched α-dodecylene, and the product was converted by means of a Friedel-Crafts reaction, using aluminum trichloride or hydrogen fluoride, to tetrapropylene benzene, which was then sulfonated and neutralized. This capability of economically producing tetrapropylene benzene sulfonates (TPS) resulted in a breakthrough for this class of surfactants, which subsequently displaced the soaps as the principal surfactant in washing and cleaning agents.

The insufficient biodegradability of TPS made it necessary to present new alkyl benzene sulfonates characterized by improved environmental behavior. These requirements are met by linear alkyl benzene sulfonates, which today are almost the only alkyl benzene sulfonates manufactured, and are referred to by the abbreviation ABS.

Linear alkyl benzene sulfonates are produced from linear alkyl benzenes, which in turn are accessible from linear olefins. For this, petroleum fractions are separated on an industrial scale, using molecular sieves, into the n-alkanes of the desired purity, and dehydrogenated to yield the n-olefins, resulting in both α- and i-olefins. The resulting olefins are then reacted with benzene in the presence of acid catalysts to yield the alkyl benzenes. The Friedel-Crafts catalyst that is selected has an influence on the isomer distribution of the resulting linear alkyl benzenes: when aluminum trichloride is used, the concentration of the 2-phenyl isomers in the mixture with the 3-, 4-, 5-, and other isomers is approximately 30 wt %; when hydrogen fluoride is used as the catalyst, on the other hand, the concentration of the 2-phenyl isomer can be decreased to approximately 20 wt %. Lastly, sulfonation of the linear alkyl benzenes is today performed on an industrial scale using oleum, sulfuric acid, or gaseous sulfur trioxide, the latter being by far the most important. Special film or tube-bundle reactors are used for sulfonation, supplying as their product a 97-wt % alkyl benzene sulfonic acid (ABSA) that is usable in the context of the present invention as an anionic surfactant acid.

A very wide variety of salts, i.e. alkyl benzene sulfonates, can be obtained from ABSAs by selecting the neutralizing medium. For economic reasons, it is preferred in this context to produce and use the alkali metal salts, and of them preferably the sodium salts, of the ABSAs. These can be described by the general formula I: 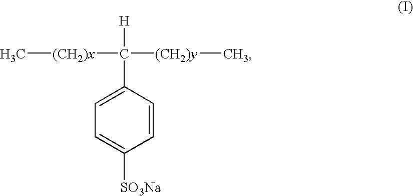
in which the sum of x and y is usually between 5 and 13. Methods according to the present invention in which C₈₋₁₆, preferably C₉₋₁₃, alkyl benzene sulfonic acids are used as the anionic surfactant in acid form are preferred. It is further preferred in the context of the present invention to use C₈₋₁₆, preferably C₉₋₁₃, alkyl benzene sulfonic acids that derive from alkyl benzenes which have a tetraline content below 5 wt % relative to the alkyl benzene. It is additionally preferred to use alkyl benzene sulfonic acids whose alkyl benzenes were produced using the HF method, so that the C₈₋₆, preferably C₉₋₁₃, alkyl benzene sulfonic acids used have a 2-phenyl isomer content below 22 wt % relative to the alkyl benzene sulfonic acid.

In summary, methods according to the present invention in which oxalic acid, malonic acid, succinic acid, glutaric acid, adipic acid, pimelic acid, azelaic acid, sebacic acid, maleic acid, fumaric acid, sorbic acid, phthalic acid, terephthalic acid, citric acid, dodecanedioic acid, stearic acid, trisodium citrate, salicylic acid, and glycolic acid, anhydrides of the aforementioned acids, or mixtures of these substances, are used as the substance to be encased, are preferred. Citric acid and/or citrates, in particular trisodium citrate, and/or citric acid anhydride, are particularly preferred for use in the method according to the present invention as the substance to be encased.

Further suitable materials that can be encased by means of the melt state in accordance with the method according to the present invention are hydrogencarbonates, in particular the alkali metal hydrogencarbonates, especially sodium and potassium hydrogencarbonate, as well as the hydrogensulfates, in particular alkali metal hydrogensulfates, especially potassium hydrogensulfate or sodium hydrogensulfate. The eutectic mixture of potassium hydrogensulfate and sodium hydrogensulfate that comprises 60 wt % NaHSO₄ and 40 wt % KHSO₄ has also proven particularly suitable. Methods according to the present invention which are characterized in that hydrogencarbonates, in particular alkali metal hydrogencarbonates, especially sodium and potassium hydrogencarbonate, and/or hydrogensulfates, in particular alkali metal hydrogensulfates, especially potassium hydrogensulfate or sodium hydrogensulfate, and/or the eutectic mixture of potassium hydrogensulfate and sodium hydrogensulfate that comprises 60 wt % NaHSO₄ and 40 wt % KHSO₄, are used as the substance to be encased are accordingly preferred.

A further substance class that can be melted and processed according to the present invention is represented by the phosphonates. These are, in particular, hydroxyalkane- or aminoalkanephosphonates. Among the hydroxyalkanephosphonates, 1-hydroxyethane-1,1-diphosphonate (HEDP) is particularly important. It is preferably used as the sodium salt, in which context the disodium salt reacts neutrally and the tetrasodium salt in alkaline fashion (pH 9). Suitable aminoalkanephosphonates are preferably ethylenediamine tetramethylenephosphonate (EDTMP), diethylenetriamine pentamethylenephosphonate (DTPMP), and their higher homologs. They are preferably used in the form of the neutrally reacting sodium salts, e.g., as the hexasodium salt of EDTMP or as the hepta- and octasodium salt of DTPMP. Of the class of phosphonates, HEDP is preferably used. The aminoalkanephosphonates furthermore possess a distinct heavy-metal binding capability. It may accordingly be preferred, especially when the medium also contains bleaches, to use aminoalkanephosphonates, in particular DTPMP, or mixtures of the aforesaid phosphonates. Methods according to the present invention in which phosphonates, preferably hydroxyalkane- or aminoalkanephosphonates, particularly preferably 1-hydroxyethane-1,1-diphosphonate (HEDP) as the di- or tetrasodium salt, and/or ethylenediamine tetramethylenephosphonate (EDTMP), diethylenetriamine pentamethylenephosphonate (DTPMP), and their higher homologs, in particular in the form of the neutrally reacting sodium salts, e.g., as the hexasodium salt of EDTMP or as the hepta- and octasodium salt of DTPMP, are used as the substance to be encased, are therefore preferred.

Further suitable materials that can be encased according to the present invention by means of the melt state are sugars. The term “sugars” in the context of the present invention identifies simple and multiple sugars, i.e. monosaccharides and oligosaccharides, in which 2 to 6 monosaccharides are joined to one another in acetal fashion. “Sugars” in the context of the present invention are therefore monosaccharides, disaccharides, trisaccharides, tetra-, penta-, and hexasaccharides.

Monosaccharides are linear polyhydroxyaldehydes (aldoses) or polyhydroxyketones (ketoses). They usually have a chain length of five (pentoses) or six (hexoses) carbon atoms. Monosaccharides having more (heptoses, octoses, etc.) or fewer (tetroses) carbon atoms are relatively rare. Monosaccharides in some cases have a large number of asymmetrical carbon atoms. For a hexose having four asymmetrical carbon atoms, this results in a total of 24 stereoisomers. The orientation of the OH group on the highest-numbered asymmetrical carbon atom in the Fischer projection divides the monosaccharides into D- and L-configured series. In the naturally occurring monosaccharides, the D-configuration is far more common. Monosaccharides form intramolecular hemiacetals when possible, thus resulting in ring-shaped structures of the pyran type (pyranoses) and furan type (furanoses). Smaller rings are unstable; larger rings survive only in aqueous solutions. Cyclization results in a further asymmetrical carbon atom (the so-called anomeric carbon atom), that once again doubles the number of possible stereoisomers. This is expressed by the prefixes α- and β-. The formation of the hemiacetals is a dynamic process that depends on a variety of factors such as temperature, solvent, pH, etc. In most cases mixtures of the two anomeric forms are present, in some cases also as mixtures of the furanose and pyranose forms.

Monosaccharides usable as sugars in the context of the present invention are, for example, the tetroses D(−)-erythrose and D(−)-threose as well as D(−)-erythrulose, the pentoses D(−)-ribose, D(−)-ribulose, D(−)-arabinose, D(+)-xylose, D(−)-xylulose and D(−)-lyxose, and the hexoses D(+)-allose, D(+)-altrose, D(+)-glucose, D(+)-mannose, D(−)-gulose, D(−)-idose, D(+)-galactose, D(+)-talose, D(+)-psicose, D(−)-fructose, D(+)-sorbose, and D(−)-tagatose. The most important and most prevalent monosaccharides are D-glucose, D-galactose, D-mannose, D-fructose, L-arabinose, D-xylose, D-ribose, and D-2-deoxyribose.

Disaccharides are constructed from two simple monosaccharide molecules (D-glucose, D-fructose, etc.) linked by a glycoside bond. If the glycoside bond is located between the acetal carbon atoms (1 for aldoses, 2 for ketoses) of the two monosaccharides, the ring shape is then immobilized in both of the them; the sugars exhibit no mutarotation, do not react with ketone reagents, and no longer have a reducing effect (Fehling negative: trehalose or sucrose type). If, in contrast, the glycoside bond joins the acetal carbon atom of one monosaccharide with any one of the second one, the latter can then still assume the open-chain configuration and the sugar still has a reducing effect (Fehling positive: maltose type).

The most important disaccharides are sucrose (raw sugar), trehalose, lactose (milk sugar), lactulose, maltose (malt sugar), cellobiose (breakdown product of cellulose), gentobiose, melibiose, turanose, and others.

Trisaccharides are carbohydrates that are made up of three monosaccharides linked to one another in glycoside fashion, for which the incorrect designation “triose” is also occasionally encountered. Trisaccharides occur relatively seldom in nature. Examples are gentianose, kestose, maltotriose, melecitose, raffinose, and, as examples of trisaccharides containing amino sugars, streptomycin and validamycin.

Tetrasaccharides are oligosaccharides having four monosaccharide units. Examples of this class of compounds are stachyose, lychnose (galactose-glucose-fructose-galactose), and secalose (comprising four fructose units).

Saccharides from the group of glucose, fructose, sucrose, cellubiose, maltose, lactose, lactulose, ribose, and mixtures thereof, are preferred for use as sugars in the context of the present invention.

Preferred methods according to the present invention are characterized in that sugars, in particular monosaccharides, disaccharides, trisaccharides, tetra-, penta-, and/or hexasaccharides, preferably sucrose, particularly preferably Isomalt®, are used as the substance to be encased. The term “Isomalt®” refers, in the context of the present invention, to a mixture of 6-O-α-D-glucopyranosyl-D-sorbitol (1,6-GPS) and 1-O-α-D-glucopyranosyl-D-mannitol (1,1-GPM). In a preferred embodiment, the weight proportion of 1,6-GPS in terms of the total weight of the mixture is less than 57 wt %. Such mixtures can be produced industrially, for example, by enzymatic transposition of sucrose into isomaltose and subsequent catalytic hydrogenation of the resulting isomaltose, forming an odorless, colorless, crystalline solid.

A further particularly preferred material that can be encased according to the present invention is urea, the diamide of carbonic acid, which is occasionally also referred to as “carbamide” and can be described by the formula H₂N—CO—NH₂. Urea forms colorless, odorless crystals having a density of 1.335, that melt at 133° C. Urea is soluble in water, methanol, ethanol, and glycerol, with a neutral reaction. Especially mixed with other substances, urea is outstandingly suitable as a material for the method according to the present invention. For example, nonionic surfactants, fragrances, dyes, etc. can be melted and encased in large quantities together with the urea. Particularly preferred in this context as materials are mixtures of urea and nonionic surfactants, which can contain up to 50 wt % nonionic surfactant relative to the mixture. Methods preferred according to the present invention are characterized in that urea is used as the substance to be encased.

As already indicated, further active substances can be mixed into the substance to be encased, provided the mixed-in materials withstand the temperature conditions in the melt. These can be, for example, dyes and/or fragrances; nonionic surfactants or other ingredients of washing or cleaning agents are also suitable.

A description of the preferred encasing media now follows. Methods preferred according to the present invention are characterized in that polyethylene glycols (PEGs) and/or polypropylene glycols (PPGs) are used as the encasing medium, PEGs and/or PPGs having melting points from 3 to 150° C., preferably from 30 to 120° C., more preferably from 40 to 100° C., and in particular from 50 to 80° C., being preferred. Both substances that are solid at room temperature and substances that are liquid at room temperature are therefore suitable as encasing media. If the encasing media are liquid at room temperature, the resulting encased substances are then, in a preferred embodiment, coated with a second encasing medium that is solid at room temperature. It is preferable in the context of the present application, however, to use encasing media that are solid at room temperature. Particularly preferred methods are therefore characterized in that polyethylene glycols (PEGs) and/or polypropylene glycols (PPGs) are used as the encasing medium, PEGs and/or PPGs having melting points from 30 to 150° C., preferably from 30 to 120° C., more preferably from 40 to 100° C., and in particular from 50 to 80° C., being preferred

Polyethylene glycols are polymers of ethylene glycol conforming to the general formula (II)
H—(O—CH₂—CH₂)ₙ—OH (II)
where n can assume values between 1 (ethylene glycol) and several thousand. Various nomenclatures exist for polyethylene glycols, and can result in confusion. The common technical practice is to indicate the average relative molecular weight following the term “PEG”, so that “PEG 200” characterizes a polyethylene glycol having a relative molar weight of approximately 190 to approximately 210. For cosmetic ingredients a different nomenclature is used, in which the abbreviation PEG has a hyphen added to it, and the hyphen is followed directly by a number corresponding to the number n in the above formula V II. According to this nomenclature (so-called INCI nomenclature, CTFA International Cosmetic Ingredient Dictionary and Handbook, 5th Edition, The Cosmetic, Toiletry and Fragrance Association, Washington, 1997), for example, PEG-4, PEG-6, PEG-8, PEG-9, PEG-10, PEG-12, PEG-14, and PEG-16 can be used. Polyethylene glycols are available commercially, for example, under the trade names Carbowax® PEG 200 (Union Carbide), Emkapol® 200 (ICI Americas), Lipoxol® 200 MED (Huls America), Polyglycol® E-200 (Dow Chemical), Alkapol® PEG 300 (Rhône-Poulenc), Lutrol® E300 (BASF), and the corresponding trade names with higher numbers.

Polypropylene glycols (abbreviated PPG) are polymers of propylene glycol that conform to the general formula III 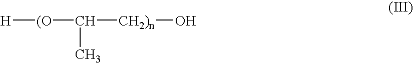
in which n can assume values between 1 (propylene glycol) and several thousand.

Nonionic surfactants, for example, are suitable as further encasing materials. With reference to what has been said above regarding the melting points of the encasing material, surfactants that have a melting point above 20° C., preferably above 25° C., particularly preferably between 25 and 60° C., and in particular between 26.6 and 43.3° C., are preferred.

Nonionic surfactants that are solid at room temperature and are preferred for use derive from the group of the alkoxylated nonionic surfactants, in particular the ethoxylated primary alcohols, and mixtures of these surfactants with structurally more-complex surfactants such as polyoxypropylene/polyoxyethylene/polyoxypropylene (PO/EO/PO) surfactants. Such (PO/EO/PO) nonionic surfactants are moreover characterized by good foaming control.

In a preferred embodiment of the present invention, the nonionic surfactant having a melting point above room temperature is an ethoxylated nonionic surfactant that has resulted from the reaction of a monohydroxyalkanol or alkyl phenol having 6 to 20 carbon atoms with preferably at least 12 mol, particularly preferably at least 15 mol, in particular at least 20 mol, of ethylene oxide per mol of alcohol or alkyl phenol.

A nonionic surfactant that is solid at room temperature and is particularly preferred for use is obtained from a straight-chain fatty alcohol having 16 to 20 carbon atoms (C₁₆₋₂₀ alcohol), preferably a C₁₈ alcohol, and at least 12 mol, preferably at least 15 mol, and in particular at least 20 mol of ethylene oxide. Of these, the so-called “narrow range ethoxylates” are particularly preferred.

Accordingly, in particularly preferred methods according to the present invention, ethoxylated nonionic surfactant(s) that was/were obtained from C₆₋₂₀ monohydroxyalkanols or C₆₋₂₀ alkyl phenols or C₁₆₋₂₀ fatty alcohols and more than 12 mol, preferably more than 15 mol, and in particular more than 20 mol ethylene oxide per mol of alcohol are used.

The nonionic surfactant preferably additionally possesses propylene oxide units in the molecule. Such PO units preferably constitute up to 25 wt %, particularly preferably up to 20 wt %, and in particular up to 15 wt % of the entire molar weight of the nonionic surfactant. Particularly preferred nonionic surfactants are ethoxylated monohydroxyalkanols or alkyl phenols that additionally comprise polyoxyethylene-polyoxypropylene block copolymer units. The alcohol or alkyl phenol portion of such nonionic surfactant molecule preferably makes up more than 30 wt %, particularly preferably more than 50 wt %, and in particular more than 70 wt % of the total molar weight of such nonionic surfactants. Preferred methods according to the present invention are characterized in that ethoxylated and propoxylated nonionic surfactants in which the propylene oxide units in the molecule constitute up to 25 wt %, preferably up to 20 wt %, and in particular up to 15 wt % of the total molar weight of the nonionic surfactant are used as the encasing material.

Additional nonionic surfactants having melting points above room temperature that are particularly preferred for use contain 40 to 70% of a polyoxypropylene/polyoxyethylene/polyoxypropylene block polymer blend, which contains 75 wt % of a reverse block copolymer of polyoxyethylene and polyoxypropylene having 17 mol ethylene oxide and 44 mol propylene oxide, and 25 w % of a block copolymer of polyoxyethylene and polyoxypropylene, initiated with trimethylol propane and containing 24 mol ethylene oxide and 99 mol propylene oxide per mol of trimethylol propane.

Nonionic surfactants that can be used with particular advantage are obtainable, for example, from Olin Chemicals under the name Poly Tergent® SLF-18.

Additional preferred nonionic surfactants usable according to the present invention conform to the formula
R¹O[CH₂CH(CH₃)O]ₓ[CH₂CH₂O]_{y}[CH₂CH(OH)R²],
in which R¹ denotes a linear or branched aliphatic hydrocarbon radical having 4 to 18 carbon atoms, or mixtures thereof; R² a linear or branched hydrocarbon radical having 2 to 26 carbon atoms, or mixtures thereof: and x denotes values between 0.5 and 1.5 and y denotes a value of at least 15.

Additional nonionic surfactants that are usable in preferred fashion are the end-group-terminated poly(oxyalkylated) nonionic surfactants of the following formula:
R¹O[CH₂CH(R³)O]ₓ[CH₂]ₖCH(OH)[CH₂]ⱼOR²
in which R¹ and R² denote linear or branched, saturated or unsaturated, aliphatic or aromatic hydrocarbon radicals having 1 to 30 carbon atoms; R³ denotes H or a methyl, ethyl, n-propyl, isopropyl, n-butyl, 2-butyl, or 2-methyl-2-butyl radical; x denotes values between 1 and 30; and k and j denote values between 1 and 12, preferably between 1 and 5. If the value of x is greater than or equal to 2, each R³ in the formula above can be different. R¹ and R² are preferably linear or branched, saturated or unsaturated, aliphatic or aromatic hydrocarbon radicals having 6 to 22 carbon atoms, radicals having 8 to 18 carbon atoms being particularly preferred. For the R³ radical, H, —CH₃, or —CH₂CH₃ are particularly preferred. Particularly preferred values for x are in the range from 1 to 20, in particular from 6 to 15.

As described above, each R³ in the formula above can be different if x≧2. The alkylene oxide unit in the square brackets can thereby be varied. If, for example, x denotes 3, the R³ radical can be selected so as to form ethylene oxide (R³═H) or propylene oxide (R³═CH₃) units, which can be joined to one another in any sequence, for example (EO)(PO)(EO), (EO)(EO)(PO), (EO)(EO)(EO), (PO)(EO)(PO), (PO)(PO)(EO), and (PO)(PO)(PO). The value of 3 for x was selected as an example here, and can certainly be larger; the range of variation increases with rising values of x, and includes e.g., a large number of (EO) groups combined with a small number of (PO) groups, or vice versa.

Particularly preferred end-group-terminated poly(oxyalkylated) alcohols of the above formula have values of k=1 and j=1, so that the formula above is simplified to
R¹O[CH₂CH(R³)O]ₓCH₂CH(OH)CH₂OR²
In the latter formula, R¹, R², and R³ are as defined above, and x denotes numbers from 1 to 30, preferably from 1 to 20, and in particular from 6 to 18. Surfactants in which the R¹ and R² radicals have 9 to 14 carbon atoms, R³ denotes H, and x assumes values from 6 to 15, are particularly preferred.

Summarizing what has just been stated, preferred methods according to the present invention are those in which end-group-terminated poly(oxyalkylated) surfactants of the formula
R¹O[CH₂CH(R³)O]ₓ[CH₂]ₖCH(OH)[CH₂]ⱼOR²
are used, in which R¹ and R³ denote linear or branched, saturated or unsaturated, aliphatic or aromatic hydrocarbon radicals having 1 to 30 carbon atoms; R³ denotes H or a methyl, ethyl, n-propyl, isopropyl, n-butyl, 2-butyl, or 2-methyl-2-butyl radical; x denotes values between 1 and 30, and k and j denote values between 1 and 12, preferably between 1 and 5, surfactants of the following type:
R¹O[CH₂CH(R³)O]ₓCH₂CH(OH)CH₂OR²
in which x denotes numbers from 1 to 30, preferably from 1 to 20, and in particular from 6 to 18, being particularly preferred.

Substances that are not water-soluble, or soluble in water only with difficulty, can also be used as encasing materials. In washing or cleaning agents, such encasings then provide for temperature-controlled release of the ingredients, while the encasings described above also release the encased material as a result of their release kinetics. In the sector of washing or cleaning agents automatic dishwashing is more suitable that textile washing as a field of application for substances having a non-water-soluble encasing because of the more pronounced temperature change during the course of a program.

In the context of non-water-soluble encasing materials, it has proven advantageous if the casing substance does not exhibit a sharply defined melting point, such as usually occurs with pure crystalline substance, but instead has a melting range that in some circumstances encompasses several degrees Celsius.

The encasing substance preferably has a melting range located between approximately 45° C. and approximately 75° C. In the present case, this means that the melting range occurs within the indicated temperature interval, and does not describe the width of the melting range. The width of the melting range is preferably at least 1° C., preferably approximately 2 to 3° C.

The aforesaid properties are fulfilled, as a rule, by so-called waxes. “Waxes” are understood to be a number of natural or artificially obtained substances that as a rule melt above 40° C. without decomposition, and just above the melting point are already relatively low in viscosity and not stringy. They exhibit a highly temperature-dependent consistency and solubility.

Waxes are divided into three groups depending on their derivation: natural waxes, chemically modified waxes, and synthetic waxes.

The natural waxes include, for example, vegetable waxes such as candellila wax, carnauba wax, Japan wax, esparto grass wax, cork wax, guaruma wax, rice seed oil wax, sugar cane wax, ouricury wax, or montan wax; animal waxes such as beeswax, shellac wax, spermaceti, lanolin (wool wax), or uropygial grease; mineral waxes such as ceresin or ozocerite (earth wax); or petrochemical waxes such as petrolatum, paraffin waxes, or microcrystalline waxes.

The chemically modified waxes include, for example, hard waxes such as montan ester waxes, sassol waxes, or hydrogenated jojoba waxes.

Synthetic waxes are usually understood to be polyalkylene waxes or polyalkylene glycol waxes. Also usable as encasing materials are compounds from other substance classes that meet the aforesaid requirements in terms of softening point. Synthetic compounds that proven suitable are, for example, higher esters of phthalic acid, in particular dicyclohexyl phthalate, which is commercially available under the name Unimoll® 66 (Bayer AG). Also suitable are synthetically produced waxes from lower carboxylic acids and fatty alcohols, for example dimyristyl tartrate, which is obtainable under the name Cosmacol® ETLP (Condea). Also usable, conversely, are synthetic or partially synthetic esters from lower alcohols with fatty acids from natural sources. This substance class contains, for example, Tegin® 90 (Goldschmidt), a glycerol monostearate-palmitate. Shellac, for example Schellack-KPS-Dreiring-SP (Kalkhoff GmbH), is also usable according to the present invention as an encasing material.

Also considered among the waxes in the context of the present invention are, for example, the so-called waxy alcohols. Waxy alcohols are higher-molecular-weight, water-insoluble fatty alcohols usually having 22 to 40 carbon atoms. The waxy alcohols occur, for example in the form of wax esters of higher-molecular-weight fatty acids (waxy acids), as a principal constituent of many natural waxes. Examples of waxy alcohols are lignoceryl alcohol (1-tetracosanol), cetyl alcohol, myristyl alcohol, or melissyl alcohol. The encasing of the solid particles encased according to the present invention can, if applicable, also contain wool wax alcohols, which are to be understood as the triterpenoid and steroid alcohols, for example lanolin, which is obtainable e.g., under the commercial designation Argowax® (Pamentier & Co.). Also usable in the context of the present invention, in at least some proportion, as a constituent of the encasing are fatty acid glycerol esters or fatty acid alkanolamides or, if applicable, also water-insoluble or poorly water-soluble polyalkylene glycol compounds.

Paraffin waxes have the advantage in the context of the present invention, as compared with the other natural waxes mentioned, that no hydrolysis of the waxes (as may be expected, for example, in the case of the waxy esters) takes place in an alkaline cleaning agent environment, since paraffin wax contains no hydrolyzable groups.

Paraffin waxes are made up principally of alkanes, as well as small proportions of iso- and cycloalkanes. The paraffin to be used according to the present invention preferably has substantially no constituents having a melting point of more than 70° C., in particularly preferred fashion more than 60° C. Portions of high-melting-point alkanes in the paraffin can, if the cleaning agent bath falls below that melting temperature, leave behind undesirable wax residues on the surfaces or items that are to be cleaned. Such wax residues generally cause the cleaned surface to have an unattractive appearance, and should therefore be avoided.

The concentration, in the paraffin wax used, of alkanes, isoalkanes, and cycloalkanes that are solid at ambient temperature (usually approximately 10 to approximately 30° C.) is preferably as high as possible. The more solid wax constituents are present in a wax at room temperature, the more usable it is in the context of the present invention. As the proportion of solid wax constituents increases, the tolerance of the encasing with regard to impact or friction against other surfaces rises, resulting in longer-lasting protection of the encased active ingredients. High proportions of oils or liquid wax constituents can result in a weakening of the encasing, so that pores are opened and the encased active ingredients are exposed to the entry of the aforesaid environmental influences.

As already mentioned earlier, a melt that is to be encased according to the present invention can be made up of multiple constituents. If the corresponding ingredients cannot be melted together with one another, for example because they exhibit phase separation phenomena, they can also be melted separately and then dripped separately into the melt of the encasing material. Corresponding methods in which multiple melts of substances to be encased are dripped simultaneously into the encasing medium are preferred.

It is preferred according to the present invention if the densities of the dripped-in melts and the density of the melt of the encasing material are different. It is particularly preferred if the density of the melt to be dripped in is equal to at least 1.1 times, preferably at least 1.2 times, and in particular at least 1.3 times the density of the melt of the encasing material.

Separation of the encased particles from the melt of the encasing material is preferably accomplished by straining the encased particles. This can be done by means of corresponding scooping devices, but preferably separation is performed continuously. The use of perforated transport belts has proven particularly successful here. Methods according to the present invention in which separation of the encased solidified melt from the melt of the encasing medium is accomplished by straining the encased particles out of the melt of the encasing material, preferably continuously, particularly preferably by means of perforated transport belts out of the melting vat by means of a draining section, are preferred embodiments of the present invention.

The thickness of the encasing (coating layer) can be influenced, as already stated, by way of the size of the melt droplets, the residence time in the melt of the encasing material, and by selection of the melting points and melting temperatures. In methods preferred according to the present invention, it is 0.1 to 2500 μm, preferably 5 to 500 μm, and in particular 10 to 200 μm.

Particularly preferred methods according to the present invention are moreover characterized in that the solidified encased substance exhibits particle sizes from 0.5 to 50 mm, preferably from 1 to 10 mm, and in particular from 1.5 to 5 mm.

A further subject of the present invention is washing or cleaning agents that contain at least one end product of a method according to the present invention.

Further ingredients of such washing or cleaning agents according to the present invention are described below.

The washing or cleaning agents or washing adjuvants according to the present invention can contain all the usual ingredients of washing or cleaning agents. These are described below.

The washing or cleaning agents according to the present invention preferably contain surfactant(s), in which context anionic, nonionic, cationic, and/or amphoteric surfactants can be used. In textile washing agents, mixtures of anionic and nonionic surfactants are preferred in terms of application engineering; the proportion of anionic surfactants should be greater than the proportion of nonionic surfactants. The total surfactant content of the washing or cleaning agent according to the present invention is preferably less than 30 wt % relative to the entire agent.

Anionic surfactants or surfactant acids as a constituent of the melt to be encased, as well as nonionic surfactants as (a constituent of) the casing or as an ingredient in the melt to be encased, have already been described above. Also usable as nonionic surfactants are preferably alkoxylated, advantageously ethoxylated, in particular primary alcohols having preferably 8 to 18 carbon atoms and an average of 1 to 12 mol ethylene oxide (EO) per mol of alcohol, in which the alcohol radical can be linear or preferably methyl-branched in the 2-position, or can contain mixed linear and methyl-branched radicals, such as those that are usually present in oxo alcohol radicals. Particularly preferred, however, are alcohol ethoxylates having linear radicals made up of alcohols of natural origin having 12 to 18 carbon atoms, e.g., from coconut, palm, tallow, or oleyl alcohol, and an average of 2 to 8 EO per mol of alcohol. The preferred ethyoxylated alcohols include, for example, C₁₂₋₁₄ alcohols having 3 EO or 4 EO, C₉₋₁₁ alcohols having 7 EO, C₁₃₋₁₅ alcohols having 3 EO, 5 EO, 7 EO, or 8 EO, C₁₂₋₁₈ alcohols having 3 EO, 5 EO, or 7 EO, and mixtures thereof, such as mixtures of C₁₂₋₁₄ alcohol having 3 EO and C₁₂₋₁₈ alcohol having 5 EO. The degrees of ethoxylation indicated represent statistical averages, which can be an integer or a fraction for a specific product. Preferred alcohol ethoxylates exhibit a narrow distribution of homologs (narrow range ethoxylates, NRE). In addition to these nonionic surfactants, fatty alcohols having more than 12 EO can also be used. Examples of these are tallow fatty alcohol having 14 EO, 25 EO, 30 EO, or 40 EO.

Also usable as further nonionic surfactants are alkyl glycosides of the general formula RO(G)ₓ, in which R denotes a primary straight-chain or methyl-branched (in particular methyl-branched in the 2-position) aliphatic radical having 8 to 22, preferably 12 to 18 carbon atoms; and G is the symbol denoting a glycose unit having 5 or 6 carbon atoms, preferably glucose. The degree of oligomerization x, which indicates the distribution of monoglycosides and oligoglycosides, is any number between 1 and 10; preferably x is between 1.2 and 1.4.

A further class of nonionic surfactants used in preferred fashion, which are used either as the only nonionic surfactant or in combination with other nonionic surfactants, is alkoxylated, preferably ethoxylated or ethoxylated and propoxylated, fatty acid alkyl esters, preferably having 1 to 4 carbon atoms in the alkyl chain, in particular fatty acid methyl esters.

Nonionic surfactants of the aminoxide type, for example N-cocalkyl-N,N-dimethylaminoxide and N-tallowalkyl-N,N-dihydroxyethylaminoxide, and the fatty acid alkanolamides, can also be suitable. The quantity of these nonionic surfactants is preferably no more than that of the ethoxylated fatty alcohols, in particular no more than half thereof.

Further suitable surfactants are polyhydroxy fatty acid amides of the following formula: 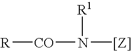
in which RCO denotes an aliphatic acyl radical having 6 to 22 carbon atoms; R¹ denotes hydrogen, an alkyl or hydroxyalkyl radical having 1 to 4 carbon atoms; and [Z] denotes a linear or branched polyhydroxyalkyl radical having 3 to 10 carbon atoms and 3 to 10 hydroxyl groups. The polyhydroxy fatty acid amides are known substances that can usually be obtained by reductive amination of a reducing sugar with ammonia, an alkylamine, or an alkanolamine, and subsequent acylation with a fatty acid, a fatty acid alkyl ester, or a fatty acid chloride.

Also belonging to the group of the polyhydroxy fatty acid amides are compounds of the following formula 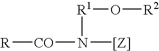
in which R denotes a linear or branched alkyl or alkylene radical having 7 to 12 carbon atoms; R¹ denotes a linear, branched, or cyclic alkyl radical or an aryl radical having 2 to 8 carbon atoms; and R² denotes a linear, branched, or cyclic alkyl radical or an aryl radical or an oxyalkyl radical having 1 to 8 carbon atoms, C₁₋₄ alkyl or phenyl radicals being preferred; and [Z1] denotes a linear polyhydroxyalkyl radical whose alkyl chain is substituted with at least two hydroxyl groups, or alkoxylated, preferably ethoxylated or propoxylated, derivatives of that radical.

[Z] is preferably obtained by reductive amination of a sugar, for example glucose, fructose, maltose, lactose, galactose, mannose, or xylose. The N-alkoxy- or N-aryloxy-substituted compounds can then be converted into the desired polyhydroxy fatty acid amides by reaction with fatty acid methyl esters in the presence of an alkoxide as catalyst.

The content of nonionic surfactants in preferred washing or cleaning agents according to the present invention that are suitable for textile washing is 5 to 20 wt %, preferably 7 to 15 wt %, and in particular 9 to 14 wt %, in each case relative to the entire agent.

In automatic dishwashing agents, low-foaming nonionic surfactants are preferably used.

In combination with the aforesaid surfactants, anionic, cationic, and/or amphoteric surfactants can also be used, these being of only subordinate importance in automatic dishwashing agents because of their foaming behavior, and being used only in quantities of less than 10 wt %, usually in fact less than 5 wt %, for example from 0.01 to 2.5 wt %, in each case relative to the agent. In washing agents, on the other hand, these surfactants are of much greater importance. The washing or cleaning agents according to the present invention can therefore also contain anionic, cationic, and/or amphoteric surfactants as surfactant components.

The agents according to the present invention can, for example, contain cationic surfactants of formulas IV, V, or VI as cationically active substances: 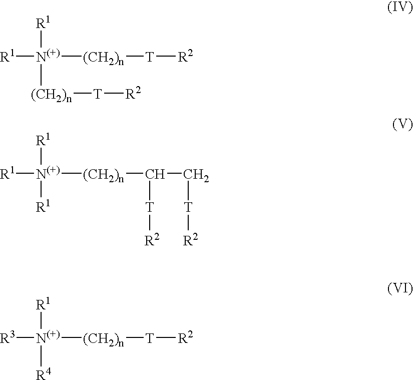
in which each R¹ group is selected, independently of one another, from C₁₋₆ alkyl, alkenyl, or hydroxyalkyl groups; each R² group, independently of one another, is selected from C₈₋₂₈ alkyl or alkenyl groups; R³=R¹ or (CH₂)ₙ-T-R²; R⁴=R¹ or R² or (CH₂)ₙ-T-R²; T=—CH₂, —O—CO—, or —CO—O—; and n is a whole number from 0 to 5.

Agents according to the present invention that are formulated in particular as conditioners contain cationic surfactant(s) of formulas (IV), (V), and/or (VI). Preferred conditioners contain 0.5 to 50 wt %, preferably 1 to 45 wt %, and in particular 2.5 to 40 wt % of at least one cationic surfactant, cationic surfactants of formula (IV) being preferred.

The anionic surfactants have already been described in detail above in their acid form. The anionic surfactant content of preferred textile washing agents according to the present invention is 5 to 25 wt %, preferably 7 to 22 wt %, and in particular 10 to 20 wt %, in each case relative to the entire agent. Cleaning agents according to the present invention for automatic dishwashing are preferably free of anionic surfactants.

In the context of the present invention, preferred agents additionally contain one or more substances from the group of the detergency builders, bleaching agents, bleach activators, enzymes, electrolytes, nonaqueous solvents, pH adjusting agents, fragrances, perfume carriers, fluorescent agents, dyes, hydrotopes, foam inhibitors, silicone oils, anti-redeposition agents, optical brighteners, graying inhibitors, shrinkage preventers, wrinkle protection agents, color transfer inhibitors, antimicrobial ingredients, germicides, fungicides, antioxidants, corrosion inhibitors, antistatic agents, ironing adjuvants, repellents and impregnating agents, stabilizing and anti-slip agents, and UV absorbers.

Phosphates, silicates, aluminum silicates (in particular zeolites), carbonates, salts of organic di- and polycarboxylic acids, and mixtures of these substances, may be mentioned in particular as detergency builders that can be contained in the agents according to the present invention.

The use of the generally known phosphates as builder substances is possible according to the present invention, provided such use in washing agents should not be avoided for environmental reasons. Cleaning agents for automatic dishwashing are usually phosphate-based, and contain preferably 30 to 70 wt %, particularly preferably 35 to 65 wt %, and in particular 45 to 60 wt % phosphate(s), in each case relative to the entire agent. Among the plurality of commercially available phosphates, the alkali metal phosphates, with particular preference for pentasodium or pentapotassium triphosphate (sodium or potassium tripolyphosphate), have the greatest significance in the washing and cleaning agent industry.

“Alkali metal phosphates” is the summary designation for the alkali-metal (in particular sodium and potassium) salts of the various phosphoric acids, in which context a distinction can be made between metaphosphoric acids (HPO₃)ₙ and orthophosphoric acid H₃PO₄, in addition to higher-molecular-weight representatives. The phosphates offer a combination of advantages: they act as alkali carriers, prevent lime deposits on machine parts and lime encrustations in fabrics, and furthermore contribute to cleaning performance. Particularly suitable are, for example, sodium dihydrogenphosphate NaH₂PO₄, disodium hydrogendiphosphate Na₂H₂P₂O₇, trisodium phosphate, tetrasodium diphosphate (sodium pyrophosphate) Na₄P₂O₇, tertiary sodium phosphate Na₃PO₄, sodium trimetaphosphate (Na₃P₃O₉) and Maddrell salt (see below), potassium dihydrogenphosphate (KH₂PO₄), dipotassium hydrogenphosphate (secondary or dibasic potassium phosphate) K₂HPO₄, tripotassium phosphate (tertiary or tribasic potassium phosphate) K₃PO₄, potassium polyphosphate (KPO₃)ₓ, potassium diphosphate (potassium pyrophosphate) K₄P₂O₇.

Condensation of NaH₂PO₄ or KH₂PO₄ yields higher-molecular-weight sodium and potassium phosphates, within which a distinction can be made between cyclic representatives (the sodium and potassium metaphosphates) and chain types (the sodium and potassium polyphosphates). For the latter in the particular, a number of designations are in use: fused or thermal phosphates, Graham salt, Kurrol's salt, and Maddrell salt. All the higher sodium and potassium phosphates are together referred to as “condensed” phosphates.

The technically important pentasodium triphosphate Na₅P₃O₁₀ (sodium tripolyphosphate) is a white, water-soluble, non-hygroscopic salt, crystallizing anhydrously or with 6H₂O, of the general formula NaO-[P(O)(ONa)-O]ₙ—Na, where n=3. Approximately 17 g of the salt containing no water of crystallization dissolves in 100 g of water at room temperature, approx. 20 g at 60° C., and approx. 32 g at 100°; after the solution is heated to 100° for two hours, approx. 8% orthophosphate and 15% disphosphate are produced by hydrolysis. In the production of pentasodium triphosphate, phosphoric acid is reacted with a soda solution or sodium hydroxide in a stoichiometric ratio, and the solution is dewatered by spraying. Like Graham salt and sodium diphosphate, pentasodium triphosphate dissolves many insoluble metal compounds (including lime soaps, etc.). Pentapotassium triphosphate K₅P₃O₁₀ (potassium tripolyphosphate) is marketed, for example, in the form of a 50 wt % solution (>23% P₂O₅, 25% K₂O). The potassium polyphosphates are widely used in the washing and cleaning agent industry. Sodium potassium tripolyphosphates also exist; these are likewise usable in the context of the present invention. They are produced, for example, when sodium trimetaphosphate is hydrolyzed with KOH:
(NaPO₃)₃+2 KOH→Na₃K₂P₃O₁₀+H₂O
These are usable according to the present invention in just the same way as sodium tripolyphosphate, potassium tripolyphosphate, or mixtures of the two; mixtures of sodium tripolyphosphate and sodium potassium tripolyphosphate, or mixtures of potassium tripolyphosphate and sodium potassium tripolyphosphate, or mixtures of sodium tripolyphosphate and potassium tripolyphosphate and sodium potassium tripolyphosphate are also usable according to the present invention.

Suitable crystalline, layered sodium silicates possess the general formula NaMSiₓO₂ₓ₊₁.H₂O, where M denotes sodium or hydrogen, x a number from 1.9 to 4, and y is a number from 0 to 20, and preferred values for x are 2, 3, or 4. Preferred crystalline sheet silicates of the formula indicated above are those in which M denotes sodium and x assumes the value 2 or 3. Both β- and δ-sodium disilicates Na₂Si₂O₅.yH₂O are particularly preferred.

Also usable are amorphous sodium silicates having a Na₂O: SiO₂ modulus of 1:2 to 1:3.3, preferably 1:2 to 1:2.8, and in particular 1:2 to 1:2.6, which are dissolution-delayed and exhibit secondary washing properties. Dissolution delay as compared with conventional amorphous sodium silicates can have been brought about in various ways, for example by surface treatment, compounding, compacting/densification, or overdrying. In the context of this invention, the term “amorphous” is also understood to mean “X-amorphous.” In other words, in X-ray diffraction experiments the silicates yield not the sharp X-ray reflections that are typical of crystalline substances, but instead at most one or more maxima in the scattered X radiation, having a width of several degree units of the diffraction angle. Particularly good builder properties can, however, very easily be obtained even if the silicate particles yield blurred or even sharp diffraction maxima in electron beam diffraction experiments. This may be interpreted to mean that the products have microcrystalline regions 10 to several hundred nm in size, values of up to a maximum of 50 nm, and in particular a maximum of 20 nm, being preferred. So-called X-amorphous silicates of this kind likewise exhibit a dissolution delay as compared with conventional water glasses. Densified/compacted amorphous silicates, compounded amorphous silicates, and overdried X-amorphous silicates are particularly preferred.

The finely crystalline synthetic zeolite containing bound water is preferably zeolite A and/or zeolite P. Zeolite MAP® (commercial product of the Crosfield Co.) is particularly preferred as zeolite P. Also suitable, however, are zeolite X as well as mixtures of A, X, and/or P. Also commercially available and preferred for use in the context of the present invention is, for example, a co-crystal of zeolite X and zeolite A (approx. 80 wt % zeolite X) that is marketed by CONDEA Augusta S.p.A. under the trade name VEGOBOND AX® and can be described by the formula
nNa₂O.(1−n)K₂O.Al₂O₃.(2−2,5)SiO₂.(3,5−5,5) H₂O
The zeolite can be used as a spray-dried powder or also as an undried stabilized suspension still moist as manufactured. In the event the zeolite is used as a suspension, it can contain small additions of nonionic surfactants as stabilizers, for example 1 to 3 wt %, relative to the zeolite, of ethoxylated C₁₂-C₁₈ fatty alcohols having 2 to 5 ethylene oxide groups, C₁₂-C₁₄ fatty alcohols having 4 to 5 ethylene oxide groups, or ethoxylated isotridecanols. Suitable zeolites exhibit an average particle size of less than 10 μm (volume distribution; measurement method: Coulter Counter), and preferably contain 18 to 22 wt %, in particular 20 to 22 wt %, of bound water.

Further important detergency builders are, in particular, the carbonates, citrates, and silicates. Trisodium citrate and/or pentasodium tripolyphosphate and/or sodium carbonate and/or sodium bicarbonate and/or gluconates and/or silicate builders from the class of the disilicates and/or metasilicates are preferred for use.

Alkali carriers can be added as further constituents. Alkali carriers are considered to be alkali metal hydroxides, alkali metal carbonates, alkali metal hydrogencarbonates, alkali metal sesquicarbonates, alkali silicates, alkali metal silicates, and mixtures of the aforesaid substances, the alkali carbonates, in particular sodium carbonate, sodium hydrogencarbonate, or sodium sesquicarbonate, being used in preferred fashion for purposes of this invention.

A builder system containing a mixture of tripolyphosphate and sodium carbonate is particularly preferred.

Also particularly preferred is a builder system containing a mixture of tripolyphosphate and sodium carbonate and sodium disilicate.

Further ingredients can additionally be added, washing, dishwashing, or cleaning agents according to the present invention that additionally contain one or more substances from the group of the acidifying agents, chelate complex-forming agents, or deposition-inhibiting polymers being preferred.

A further possible group of ingredients is represented by the chelate complex-forming agents. Chelate complex-forming agents are substances that form cyclic compounds with metal ions, a single ligand occupying more than one coordination site on a central atom, i.e. being at least “double-toothed.” In this case, therefore, normally elongated compounds are closed up into rings by forming a complex by means of an ion. The number of bound ligands depends on the coordination number of the central ion.

Common chelate complex-forming agents that are preferred in the context of the present invention are, for example, polyoxycarboxylic acids, polyamines, ethylenediamine tetraacetic acid (EDTA), and nitrilotriacetic acid (NTA). Also usable according to the present invention are complex-forming polymers, i.e. polymers that carry, either in the main chain itself or laterally thereto, functional groups that can act as ligands and react with suitable metal atoms, generally forming chelate complexes. The polymer-bound ligands of the resulting metal complexes can derive from only one macromolecule or can belong to different polymer chains. The latter case results in crosslinking of the material, provided the complex-forming polymers were not already crosslinked by means of covalent bonds.

Complexing groups (ligands) of usual complex-forming polymers are iminodiacetic acid, hydroxyquinoline, thiourea, guanidine, dithiocarbamate, hydroxamic acid, amide oxime, aminophosphoric acid, (cyclic) polyamino, mercapto, 1,3-dicarbonyl, and crown ether radicals, in some cases having very specific activities with respect to ions of various metals. Fundamental polymers of many complex-forming polymers that are also commercially important are polystyrene, polyacrylates, polyacrylonitriles, polyvinyl alcohols, polyvinyl pyridines, and polyethylene imines. Natural polymers such as cellulose, starch, or chitin are also complex-forming polymers. The latter can additionally be equipped with further ligand functionalities by polymer-analogous conversions.

Particularly preferred in the context of the present invention are washing or cleaning agents that contain one or more chelate complex formers from the groups of the
(i) polycarboxylic acids in which the sum of the carboxyl and (if applicable) hydroxyl groups is at least 5;
(ii) nitrogen-containing mono- or polycarboxylic acids;
(iii) geminal diphosphonic acids;
(iv) aminophosphonic acids
(v) phosphonopolycarboxylic acids
(vi) cyclodextrins,
   in quantities above 0.1 wt %, preferably above 0.5 wt %, particularly preferably above 1 wt %, and in particular above 2.5 wt %, in each case relative to the weight of the agent.

All complex formers of the existing art can be used in the context of the present invention. They can belong to different chemical groups. The following are preferably used, individually or mixed with one another:

a) polycarboxylic acids in which the sum of the carboxyl and (if applicable) hydroxyl groups is at least 5, such as gluconic acid;

b) nitrogen-containing mono- or polycarboxylic acids such as ethylenediamine tetraacetic acid (EDTA), N-hydroxyethyl ethylenediamine tetraacetic acid, diethylenediamine pentaacetic acid, hydroxyethylimino diacetic acid, nitridodiacetic acid 3-propionic acid, isoserine diacetic acid, N,N-di-(p-hydroxyethyl) glycine, N-(1,2-dicarboxy-2-hydroxyethyl) glycine, N-(1,2-dicarboxy-2-hydroxyethyl) aspartic acid, or nitrilotriacetic acid (NTA);

c) geminal diphosphonic acids such as 1-hydroxyethane-1,1-diphosphonic acid (HEDP), its higher homologs having up to 8 carbon atoms, and hydroxy- or amino-group-containing derivatives thereof, and 1-aminoethane-1,1-diphosphonic acid, its higher homologs having up to 8 carbon atoms, and hydroxy- or amino-group-containing derivatives thereof;

d) aminophosphonic acids, such as ethylenediamine tetra(methylphosphonic acid), diethylenetriamine penta(methylenephosphonic acid), or nitrilotri(methylenephosphonic acid);

e) phosphonopolycarboxylic acids such as 2-phosphonobutane-1,2,4-tricarboxylic acid; and

f) cyclodextrins.

In the context of this patent application, polycarboxylic acids (a) are understood as carboxylic acids (including monocarboxylic acids) in which the sum of the carboxyl and hydroxyl groups contained in the molecule is at least 5. Complex formers from the group of the nitrogen-containing polycarboxylic acids, in particular EDTA, are preferred. At the alkaline pH values of the treatment solutions necessary according to the present invention, these complex formers are present at least in part as anions. It is immaterial whether they are introduced in the form of the acids or in the form of salts. If they are used as salts, alkali, ammonium, or alkylammonium salts, in particular sodium salts, are preferred.

Deposition-inhibiting polymers can likewise be contained in the agents according to the present invention. These substances, which can have a variety of chemical structures, derive e.g., from the groups of the low-molecular-weight polyacrylates having molar weights between 1000 and 20,000 dalton, polymers having molar weights below 15,000 dalton being preferred.

Deposition-inhibiting polymers can also exhibit co-building properties. Polycarboxylates/polycarboxylic acids, polymeric polycarboxylates, aspartic acid, polyacetals, dextrins, further organic co-builders (see below), and phosphonates can be used in particular in the automatic dishwashing agents according to the present invention. These substance classes are described below.

Usable organic builder substances are, for example, the polycarboxylic acids usable in the form of their sodium salts, “polycarboxylic acids” being understood as those carboxylic acids that carry more than one acid function. These are, for example, citric acid, adipic acid, succinic acid, glutaric acid, malic acid, tartaric acid, maleic acid, fumaric acid, sugar acids, aminocarboxylic acids, nitrilotriacetic acid (NTA), provided such use is not objectionable for environmental reasons, as well as mixtures thereof. Preferred salts are the salts of the polycarboxylic acids such as citric acid, adipic acid, succinic acid, glutaric acid, tartaric acid, sugar acids, and mixtures thereof. These are preferably used coated in accordance with the method according to the present invention.

The acids per se can also be used. The acids typically also possess, in addition to their builder effect, the property of an acidifying component, and thus serve also to establish a lower and milder pH for washing or cleaning agents. Worthy of mention in this context are, in particular, citric acid, succinic acid, glutaric acid, adipic acid, gluconic acid, and any mixtures thereof.

Further polymeric polycarboxylates are suitable as builders or deposition inhibitors; these are, for example, the alkali metal salts of polyacrylic acid or polymethacrylic acid, for example those having a relative molecular weight of 500 to 70,000 g/mol.

The molar weights indicated for the polymeric polycarboxylates are, for purposes of this document, weight-averaged molar weights M_{w} of the respective acid form that were determined in principle by means of gel permeation chromatography (GPC), a UV detector having been used. The measurement was performed against an external polyacrylic acid standard that, because of its structural relationship to the polymers being investigated, yielded realistic molecular weight values. These indications deviate considerably from the molecular weight indications in which polystyrene sulfonic acids are used as the standard. The molar weights measured against polystyrene sulfonic acids are usually much higher than the molar weights indicated in this document.

Suitable polymers are, in particular, polyacrylates that preferably have a molecular weight from 500 to 20,000 g/mol. Because of their superior solubility, of this group the short-chain polyacrylates that have molar weights from 1000 to 10,000 g/ml, and particularly preferably from 1000 to 4000 g/mol, may in turn be preferred.

It is particularly preferred to use in the agents according to the present invention both polyacrylates and copolymers of unsaturated carboxylic acids, sulfonic acid group-containing monomers, and optionally further ionic or nonionogenic monomers. The sulfonic acid group-containing copolymers are described in detail below.

Copolymeric polycarboxylates, in particular those of acrylic acid with methacrylic acid and of acrylic acid or methacrylic acid with maleic acid, are also suitable. Copolymers of acrylic acid with maleic acid that contain 50 to 90 wt % acrylic acid and 50 to 10 wt % maleic acid have proven particularly suitable. Their relative molecular weight, relative to free acids, is generally 2000 to 70,000 g/mol, preferably 20,000 to 50,000 g/mol, and in particular 30,000 to 40,000 g/mol.

The (co)polymeric polycarboxylates can be used either as a powder or as an aqueous solution. The (co)polymeric polycarboxylate content of the agents is preferably 0.5 to 20 wt %, in particular 3 to 10 wt %.

Also particularly preferred are biodegradable polymers made up of more than two different monomer units, for example those that contain salts of acrylic acid and maleic acid, as well as vinyl alcohol or vinyl alcohol derivatives, as monomers, or that contain salts of acrylic acid and 2-alkylallylsulfonic acid, as well as sugar derivatives, as monomers. Further preferred copolymers are those that have, as monomers, preferably acrolein and acrylic acid/acrylic acid salts, or acrolein and vinyl acetate.

Also to be mentioned as further preferred builder substances are polymeric aminodicarboxylic acids, their salts, or their precursor substances. Particularly preferred are polyaspartic acids and their salts and derivatives, which have not only co-builder properties but also a bleach-stabilizing effect.

Other suitable builder substances are polyacetals, which can be obtained by reacting dialdehydes with polyol carboxylic acids that have 5 to 7 carbon atoms and at least 3 hydroxyl groups. Preferred polyacetals are obtained from dialdehydes such as glyoxal, glutaraldehyde, terephthalaldehyde, and mixtures thereof, and from polyol carboxylic acids such as gluconic acid and/or glucoheptonic acid.

Other suitable organic builder substances are dextrins, for example oligomers or polymers of carbohydrates, which can be obtained by partial hydrolysis of starches. The hydrolysis can be performed in accordance with usual, e.g., acid- or enzyme-catalyzed, methods. Preferably these are hydrolysis products having average molar weights in the range from 400 to 500,000 g/mol. A polysaccharide having a dextrose equivalent (DE) in the range from 0.5 to 40, in particular from 2 to 30, is preferred, DE being a common indicator of the reducing effect of a polysaccharide as compared with dextrose, which possesses a DE of 100. Also usable are maltodextrins having a DE between 3 and 20, and dry glucose syrups having a DE between 20 and 37, as well as so-called yellow dextrins and white dextrins having higher molar weights in the range from 2000 to 30,000 g/mol.

The oxidized derivatives of such dextrins are their reaction products with oxidizing agents that are capable of oxidizing at least one alcohol function of the saccharide ring to the carboxylic acid function. A product oxidized at C₆ of the saccharide ring can be particularly advantageous.

Oxydisuccinates and other derivatives of disuccinates, preferably ethylenediamine disuccinate, are also additional suitable co-builders. Ethylenediamine N,N′-disuccinate (EDDS) is used here preferably in the form of its sodium or magnesium salts. Also preferred in this context are glycerol disuccinates and glycerol trisuccinates. Suitable utilization quantities in zeolite-containing and/or silicate-containing formulations are 3 to 15 wt %.

Other usable organic co-builders are, for example, acetylated hydroxycarboxylic acids and their salts, which can optionally also be present in lactone form and which contain at least 4 carbon atoms and at least one hydroxy group, as well as a maximum of two acid groups.

A further substance class having co-builder properties is represented by the phosphonates. These are, in particular, hydroxyalkane- and aminoalkanephosphonates. Among the hydroxyalkanephosphonates, 1-hydroxyethane-1,1-diphosphonate (HEDP) is particularly important as a co-builder. It is preferably used as the sodium salt, in which context the disodium salt reacts neutrally and the tetrasodium salt in alkaline fashion (pH 9). Suitable aminoalkanephosphonates are preferably ethylenediamine tetramethylenephosphonate (EDTMP), diethylenetriamine pentamethylenephosphonate (DTPMP), and their higher homologs. They are preferably used in the form of the neutrally reacting sodium salts, e.g., as the hexasodium salt of EDTMP or as the hepta- and octasodium salt of DTPMP. Of the class of phosphonates, HEDP is preferably used as a builder. The aminoalkanephosphonates furthermore possess a pronounced heavy-metal binding capability. It may accordingly be preferred, especially when the agent also contains bleaches, to use aminoalkanephosphonates, in particular DTPMP, or mixtures of the aforesaid phosphonates These substances as well are preferably used coated in accordance with the method according to the present invention.

In addition to the substances from the aforesaid substance classes, the agents according to the present invention can contain further usual ingredients of washing, dishwashing, or cleaning agents, in which context bleaching agents, bleach activators, enzymes, silver protection agents, dyes, and fragrances are of particular importance. These substance are described below.

Of the compounds serving as bleaching agents that yield H₂O₂ in water, sodium percarbonate has particular importance. Additional usable bleaching agents are, for example, sodium perborate tetrahydrate and sodium perborate monohydrate, peroxypyrophosphates, citrate perhydrates, and peracid salts or peracids that yield H₂O₂, such as perbenzoates, peroxyphthalates, diperazelaic acid, phthaloimino peracid, or diperdodecanedioic acid.

To achieve an improved bleaching effect when washing at temperatures of 60° C. and below, bleach activators can be incorporated into washing and cleaning agents according to the present invention. Compounds that, under perhydrolysis conditions, yield aliphatic peroxycarboxylic acids having preferably 1 to 10 carbon atoms, in particular 2 to 4 carbon atoms, and/or optionally substituted perbenzoic acid, can be used as bleach activators. Substances that carry the O— and/or N-acyl groups having the aforesaid number of carbon atoms, and/or optionally substituted benzoyl groups, are suitable. Multiply acylated alkylenediamines, in particular tetraacetyl ethylenediamine (TAED), acylated triazine derivatives, in particular 1,5-diacetyl-2,4-dioxyhexahydro-1,3,5-triazine (DADHT), acylated glycolurils, in particular tetraacetyl glycoluril (TAGU), N-acylimides, in particular N-nonanoyl succinimide (NOSI), acylated phenol sulfonates, in particular n-nonanoyl or isononanoyl oxybenzene sulfonate (n- and iso-NOBS), carboxylic acid anhydrides, in particular phthalic acid anhydride, acylated polyvalent alcohols, in particular triacetin, ethylene glycol diacetate, and 2,5-diacetoxy-2,5-dihydrofuran, are preferred.

In addition to or instead of the conventional bleach activators, so-called bleach catalysts can also be incorporated. These substances are bleach-enhancing transition metal salts or transition metal complexes such as, for example, Mn, Fe, Co, Ru, or Mo salt complexes or carbonyl complexes. Mn, Fe, Co, Ru, Mo, Ti, V, and Cu complexes having nitrogen-containing tripod ligands, as well as Co, Fe, Cu, and Ru ammine complexes, are also applicable as bleach catalysts.

Agents according to the present invention can contain enzymes in order to enhance washing or cleaning performance, all enzymes established in the existing art for those purposes being usable in principle. These include, in particular, proteases, amylases, lipases, hemicellulases, cellulases, or oxidoreductases, as well as preferably mixtures thereof. These enzymes are, in principle, of natural origin; improved variants based on the natural molecules are available for use in washing and cleaning agents and are correspondingly preferred for use. Agents according to the present invention contain enzymes preferably in total quantities from 1×10⁻⁶ to 5 wt %, relative to active protein. The protein concentration can be determined with known methods, for example the bicinchoninic acid method (BCA: 2,2′-diquinolyl-4,4′-dicarboxylic acid), or the biuret method.

Among the proteases, those of the subtilisin type are preferred. Examples thereof are the subtilisins BPN′ and Carlsberg, protease PB92, subtilisins 147 and 309, the alkaline protease from Bacillus lentus, subtilisin DY, and the enzymes (to be classified, however, as subtilases rather than as subtilisins in the strict sense) thermitase, proteinase K, and proteases TW3 and TW7. Subtilisin Carlsberg is obtainable in further developed form under the trade name Alcalase® from Novozymes A/S, Bagsvaerd, Denmark. Subtilisins 147 and 309 are marketed by Novozymes under the trade names Esperase® and Savinase®, respectively. The variants listed under the designation BLAP® are derived from the protease from Bacillus lentus DSM 5483. Additional usable proteases derive from various Bacillus sp. and B. gibsonii.

Other usable proteases are, for example, the enzymes obtainable under the trade names Durazym®, Relase®, Everlase®, Nafizym, Natalase®, Kannase®, and Ovozymes® from Novozymes, under the trade names Purafect®, Purafect® O×P and Properase® from Genencor, under the trade name Protosol® from Advanced Biochemicals Ltd., Thane, India, under the trade name Wuxi® from Wuxi Snyder Bioproducts Ltd., China, under the trade names Proleather® and Protease P® from Amano Pharmaceuticals Ltd., Nagoya, Japan, and under the designation Proteinase K-16 from Kao Corp., Tokyo, Japan.

Examples of amylases usable according to the present invention are the α-amylases from Bacillus licheniformis, from B. amyloliquefaciens, or from B. stearothermophilus, and their further developments improved for use in washing and cleaning agents. The enzyme from B. licheniformus is available from Novozymes under the name Termamyl®, and from Genencor under the name Purastar® ST. Further developed products of these α-amylases are available from Novozymes under the trade names Duramyl® and Termamyl® ultra, from Genencor under the name Purastar® 0×Am, and from Daiwa Seiko Inc., Tokyo, Japan, as Keistase®. The α-amylase from B. amyloliquefaciens is marketed by Novozymes under the name BAN®, and derived variants of the α-amylase from B. stearothermophilus are marketed, again by Novozymes, under the names BSG® and Novamyl®.

Additionally to be highlighted for this purpose are the α-amylase from Bacillus sp. A 7-7 (DSM 12368) and the cyclodextrin-glucanotransferase (CGTase) from B. agaradherens (DSM 9948); also those that belong the sequence space of α-amylases. Fusion products of the aforesaid molecules are likewise usable.

The further developments of the α-amylase from Aspergillus niger and A. oryzae, obtainable from Novozymes under the trade names Fungamyl®, are also suitable. A further commercial product is, for example, Amylase-LT®.

Agents according to the present invention can contain lipases or cutinases, in particular because of their triglyceride-cleaving activities but also in order to generate peracids in situ from suitable precursors. These include, for example, the lipases obtainable originally from Humicola lanuginosa (Thermomyces lanuginosus) or further developed lipases, in particular those having the D96L amino acid exchange. They are marketed, for example, by Novozymes under the trade names Lipolase®, Lipolase® Ultra, LipoPrime®, Lipozyme®, and Lipex®. The cutinases that were originally isolated from Fusarium solani pisi and Humicola insolens are moreover usable. Usable lipases are likewise obtainable from Amano under the designations Lipase CE®, Lipase P®, Lipase B®, or Lipase CES®, Lipase AKG®, Bacillis sp. Lipase®, Lipase AP®, Lipase M-AP®, and Lipase AML®. The lipases and cutinases from, for example, Genencor, whose starting enzymes were originally isolated from Pseudomonas mendocina and Fusarium solanii, are usable. To be mentioned as further important commercial products are the preparations M1 Lipase® and Lipomax® originally marketed by Gist-Brocades, and the enzymes marketed by Meito Sangyo KK, Japan, under the names Lipase MY-30®, Lipase OF®, and Lipase PL®, as well as the Lumafast® product of Genencor.

Agents according to the present invention can, especially if they are intended for the treatment of textiles, contain cellulases, depending on the purpose as pure enzymes, as enzyme preparations, or in the form of mixtures in which the individual components advantageously complement one another in terms of their various performance aspects. These performance aspects include, in particular, contributions to primary washing performance, the secondary washing performance of the agent (anti-redeposition effect or graying inhibition), and brightening (fabric effect), or even exertion of a “stone-washed” effect.

A usable fungus-based cellulase preparation rich in endoglucanase (EG), and its further developments, are offered by Novozymes under the trade name Celluzyme®. The products Endolase® and Carezyme®, likewise obtainable from Novozymes, are based on the 50 kD EG and 43 kD EG, respectively, from H. insolens DSM 1800. Further possible commercial products of this company are Cellusoft® and Renozyme®. Cellulases are also usable, for example the 20 kD EG from Melanocarpus that is available from AB Enzymes, Finland, under the trade names Ecostone® and Biotouch®. Other commercial products of AB Enzymes are Econase® and Ecopulp®. Other suitable cellulases derive from Bacillus sp. CBS 670.93 and CBS 669.83, the one from Bacillus sp. CBS 670.93 being obtainable from Genencor under the trade name Puradax®. Other commercial products of Genencor are “Genencor detergent cellulase L” and IndiAge® Neutra.

Agents according to the present invention can contain further enzymes that are grouped under the term “hemicellulases.” These include, for example, mannanases, xanthanylases, pectinylases (=pectinases), pectinesterases, pectatylases, xyloglucanases (=xylanases), pullulanases, and β-glucanases. Suitable mannanases are obtainable, for example, under the names Gamanase® and Pektinex AR® from Novozymes, under the name Rohapec® B1 L from AB Enzymes, and under the name Pyrolase® from Diversa Corp., San Diego, Calif., USA. A β-glucanase from a B. alcalophilus is likewise suitable. The β-glucanase obtained from B. subtilis is available under the name Cereflo® from Novozymes.

To enhance the bleaching effect, washing and cleaning agents according to the present invention can contain oxidoreductases, for example oxidases, oxygenases, catalases, peroxidases such as halo-, chloro-, bromo-, lignin, glucose, or manganese peroxidases, dioxygenases, or laccases (phenoloxidases, polyphenoloxidases). Suitable commercial products that may be mentioned are Denilite® 1 and 2 of Novozymes. Advantageously, preferably organic, particularly preferably aromatic compounds that interact with the enzymes are additionally added in order to enhance the activity of the relevant oxidoreductases (enhancers) or, if there is a large difference in redox potentials between the oxidizing enzymes and the dirt particles, to ensure electron flow (mediators).

The enzymes used in the agents according to the present invention derive either originally from microorganisms, for example the genera Bacillus, Streptomyces, Humicola, or Pseudomonas, and/or are produced by suitable microorganisms in accordance with biotechnological methods known per se, for example by transgenic expression hosts of Bacillus genera or filamentous fungi.

Purification of the relevant enzymes is favorably accomplished by way of methods established per se, for example by precipitation, sedimentation, concentration, filtration of the liquid phases, microfiltration, ultrafiltration, the action of chemicals, deodorization, or suitable combinations of these steps.

Agents according to the present invention can have the enzymes added to them in any form established according to the existing art. These include, for example, the solid preparations obtained by granulation, extrusion, or lyophilization or, especially in the case of liquid or gelled agents, solutions of the enzymes, advantageously as concentrated as possible and anhydrous and/or with stabilizers added.

Alternatively, the enzymes can be encapsulated for both the solid and the liquid administration form, for example by spray-drying or extrusion of the enzyme solution together with a preferably natural polymer, or in the form of capsules, for example ones in which the enzyme is enclosed e.g., in a solidified gel, or in those of the core-shell type, in which an enzyme-containing core is covered with a protective layer impermeable to water, air, and/or chemicals. Further ingredients, for example stabilizers, emulsifiers, pigments, bleaching agents, or dyes, can additionally be applied in superimposed layers. Such capsules are applied in accordance with methods known per se, for example by vibratory or rolling granulation or in fluidized-bed processes. Such granulated materials are advantageously low in dust, e.g., as a result of the application of polymer film-forming agents, and are stable in storage thanks to the coating.

It is additionally possible to package two or more enzymes together, so that a single granulated material exhibits several enzyme activities.

A protein and/or enzyme contained in an agent according to the present invention can be protected, especially during storage, against damage such as, for example, inactivation, denaturing, or decomposition, e.g., resulting from physical influences, oxidation, or proteolytic cleavage. An inhibition of proteolysis is particularly preferred in the context of microbial recovery of the proteins and/or enzymes, in particular when the agents also contain proteases. Agents according to the present invention can contain stabilizers for this purpose; the provision of such agents represents a preferred embodiment of the present invention.

Reversible protease inhibitors are one group of stabilizers. Benzamidine hydrochloride, borax, boric acids, boronic acids, or their salts or esters are often used, among them principally derivatives having aromatic groups, e.g., ortho-substituted, meta-substituted, and para-substituted phenylboronic acids, or their salts or esters. Peptide aldehydes, i.e. oligopeptides having a reduced C terminus, are also suitable. Ovomucoid and leupeptin may be mentioned as peptide protease inhibitors; an additional option is the creation of fusion proteins from proteases and peptide inhibitors.

Further enzyme stabilizers are aminoalcohols such as mono-, di-, triethanol- and -propanolamine and mixtures thereof, aliphatic carboxylic acids up to C₁₂ such as succinic acid, other dicarboxylic acids, or salts of the aforesaid acids. End-group-terminated fatty acid amide alkoxylates are also suitable. Certain organic acids used as builders are additionally capable of stabilizing a contained enzyme.

Lower aliphatic alcohols, but principally polyols, for example glycerol, ethylene glycol, propylene glycol, or sorbitol, are other frequently used enzyme stabilizers. Diglycerol phosphate also protects against denaturing due to physical influences. Calcium salts are likewise used, for example calcium acetate or calcium formate, and magnesium salts.

Polyamide oligomers or polymeric compounds such as lignin, water-soluble vinyl copolymers, or cellulose ethers, acrylic polymers, and/or polyamides stabilize the enzyme preparation with respect to physical influences or pH fluctuations, inter alia. Polyamine-N-oxide-containing polymers act simultaneously as enzyme stabilizers and as color transfer inhibitors. Other polymeric stabilizers are the linear C₈-C₁₈ polyoxyalkylenes. Alkyl polyglycosides can stabilize the enzymatic components of the agent according to the present invention, and even improve its performance. Crosslinked nitrogen-containing compounds perform a dual function as soil-release agents and enzyme stabilizers.

Reducing agents and antioxidants increase the stability of the enzymes with respect to oxidative breakdown. Sulfur-containing reducing agents are likewise known. Other examples are sodium sulfite and reducing sugars.

Combinations of stabilizers are preferably used, for example made up of polyols, boric acid and/or borax, the combination of boric acid or borate, reducing salts, and succinic acid or other dicarboxylic acids, or the combination of boric acid or borate with polyols or polyamino compounds and with reducing salts. The effect of peptide aldehyde stabilizers is increased by the combination with boric acid and/or boric acid derivatives and polyols, and further enhanced by the additional use of divalent cations, for example calcium ions.

Cleaning agents according to the present invention for automatic dishwashing can contain corrosion inhibitors to protect the machine or the items being washed; silver protection agents, in particular, are especially important in the field of automatic dishwashing. Known substances of the existing art are usable. In general, silver protection agents can be selected principally from the group of the triazoles, benzotriazoles, bisbenzotriazoles, aminotriazoles, alkylaminotriazoles, and the transition metal salts or complexes. It is particularly preferred to use benzotriazole and/or alkylaminotriazole. Cleaner formulations moreover often comprise agents containing active chlorine, which agents can greatly decrease the corrosion of silver surfaces. In chlorine-free cleaners, oxygen- and nitrogen-containing organic redox-active compounds are used in particular, such as di- and trivalent phenols, e.g., hydroquinone, catechol, hydroxyhydroquinone, gallic acid, phloroglucine, pyrogallol, and derivatives of these classes of compounds. Salt-like and complex-like inorganic compounds, for example salts of the metals Mn, Ti, Zr, Hf, V, Co, and Ce, are also often used. Preferred in this context are the transition metal salts that are selected from the group of the manganese and/or cobalt salts and/or complexes, in particularly preferred fashion the cobalt(ammine) complexes, cobalt(acetate) complexes, cobalt(carbonyl) complexes, the chlorides of cobalt or manganese, and manganese sulfate. Zinc compounds can also be used to prevent corrosion of the items being washed.

A large number of very varied salts from the group of the inorganic salts can be used as electrolytes. Preferred cations are the alkali and alkaline earth metals, preferred anions are the halides and sulfates. In terms of production engineering, the use of NaCl or MgCl₂ in the agents according to the present invention is preferred. The concentration of electrolytes in the agents according to the present invention is usually 0.5 to 5 wt %.

In order to bring the pH of the agents according to the present invention into the desired range, the use of pH adjusting agents may be indicated. All known acids and bases are usable here, provided their use is not prohibited for environmental or applications-engineering reasons, or for reasons of consumer safety. The quantity of these adjusting agents usually does not exceed 5 wt % of the entire formulation.

Appropriate foam inhibitors that can be used in the agents according to the present invention are, for example, soaps, paraffins, or silicone oils, which optionally can be applied onto carrier materials. For suitable anti-redeposition agents (also referred to as soil repellents) packaged according to the present invention as textile washing agents, nonionic cellulose ethers such as methylcellulose and methylhydroxypropylcellulose having a 15 to 30 wt % concentration of methoxy groups and a 1 to 15 wt % concentration of hydroxypropyl groups, in each case relative to the nonionic cellulose ethers, can be used, as well as the polymers, known from the existing art, of phthalic acid and/or terephthalic acid or their derivatives, in particular polymers of ethylene terephthalates and/or polyethylene glycol terephthalates or anionically and/or nonionically modified derivatives thereof. Of these, the sulfonated derivatives of the phthalic acid and terephthalic acid polymers are particularly preferred.

Optical brighteners (so-called “whiteners”) can be added to the agents according to the present invention prepared according to the present invention as textile washing agents, in order to eliminate graying and yellowing of the treated textiles. These substances are absorbed onto the fibers and cause a brightening and simulated bleaching effect by converting invisible ultraviolet radiation into visible longer-wave light; the ultraviolet light absorbed from sunlight is radiated as a weakly bluish fluorescence, combining with the yellow tint of the grayed or yellowed laundry to yield pure white. Suitable compounds derive, for example, from the substance class of the 4,4′-diamino-2,2′-stilbenedisulfonic acids (flavonic acids), 4,4′-distyrl biphenylene, methyl umbelliferones, cumarins, dihydroquinolinones, 1,3-diarylpyrazolines, naphthalic acid imides, benzoxazole, benzisoxazole, and benzimidazole systems, and the pyrene derivatives substituted with heterocycles. The optical brighteners are usually used in quantities between 0.05 and 0.3 wt %, relative to the complete agent.

The purpose of graying inhibitors is to keep dirt released from the fibers suspended in the bath, thus preventing the dirt from redepositing. Water-soluble colloids, usually organic in nature, are suitable for this, for example size, gelatins, salts of ether sulfonic acids of starch or cellulose, or salts of acid sulfuric acid esters of cellulose or starch. Water-soluble polyamides containing acid groups are also suitable for this purpose. Soluble starch preparations, and starch products other than those mentioned above, can also be used, e.g., degraded starch, aldehyde starches, etc. Polyvinylpyrrolidone is also usable. It is preferred, however, to use cellulose ethers such as carboxymethylcellulose (Na salt), methylcellulose, hydroxyalkylcellulose, and mixed ethers such as methylhydroxyethylcellulose, methylhydroxypropylcellulose, methylcarboxylmethylcellulose, and mixtures thereof, in quantities from 0.1 to 5 wt % relative to the agent.

The agents according to the present invention can also be provided with further additional benefits. In this context it is possible to formulate, for example for agents packaged as textile washing agents, color transfer-inhibiting compositions, agents having an “anti-gray” formula, agents that facilitate ironing, agents with a particular fragrance release, agents having improved dirt detachment or prevention of soil redeposition, antibacterial agents, UV protection agents, color-brightening agents, etc. Some examples are explained below:

Because textile fabrics, in particular those made of rayon, viscose, cotton, and mixtures thereof, can tend to wrinkle because the individual fibers are sensitive to bending, kinking, compression, and squeezing transversely to the fiber direction, the agents according to the present invention can contain synthetic wrinkle-prevention agents. These include, for example, synthetic products based on fatty acids, fatty acid esters, fatty acid amides, fatty acid alkylol esters, fatty acid alkylolamides, or fatty alcohols that are usually reacted with ethylene oxide, or products based on lecithin or modified phosphoric acid esters.

The agents can contain antioxidants in order to prevent undesired changes, caused by the action of oxygen and other oxidative processes, to the agents and/or the treated textiles. This class of compounds includes, for example, substituted phenols, hydroquinones, catechols, and aromatic amines, as well as organic sulfides, polysulfides, dithiocarbamates, phosphites, and phosphonates.

Increased wearing comfort can result from the additional use of antistatic agents, which are additionally incorporated into the agents according to the present invention. Antistatic agents increase the surface conductivity and thus improve the dissipation of charges that have formed. External antistatic agents are usually substances having at least one hydrophilic molecule ligand, and form a more or less hygroscopic film on the surfaces. These usually surface-active antistatic agents can be subdivided into nitrogen-containing (amines, amides, quaternary ammonium compounds), phosphorus-containing (phosphoric acid esters), and sulfur-containing antistatic agents (alkylsulfonates, alkyl sulfates). Lauryl (or stearyl) dimethylbenzylammonium chlorides are suitable as antistatic agents for textiles or as an additive to washing agents, a brightening effect additionally being achieved.

In order to improve the water absorption capability and rewettability of the treated textiles and to facilitate ironing of the treated textiles, silicone derivatives, for example, can be used in the agents according to the present invention. These additionally improve the rinsing behavior of the agents according to the present invention as a result of their foam-inhibiting properties. Preferred silicone derivatives are, for example, polydialkyl or alkylaryl siloxanes in which the alkyl groups have one to five carbon atoms and are entirely or partly fluorinated. Preferred silicones are polydimethyl siloxanes, which optionally can be derivatized and are then aminofunctional or quaternized or have Si—OH, Si—H, and/or Si—Cl bonds. The viscosities of the preferred silicones at 25° C. are in the range between 100 and 100,000 centistokes; the silicones can be used in quantities between 0.2 and 5 wt % relative to the entire agent.

Lastly, the agents according to the present invention can also contain UV absorbers that are absorbed onto the treated textiles and improve the light-fastness of the fibers. Compounds that exhibit these desired properties are, for example, the compounds that act by radiationless deactivation, and derivatives of benzophenone having substituents in the 2- and/or 4-position. Also suitable are substituted benzotriazoles, acrylates phenyl-substituted in the 3-position (cinnamic acid derivatives) optionally having cyano groups in the 2-position, salicylates, organic Ni complexes, and natural substances such as umbelliferone and endogenous urocanic acid.

1. A method for encasing at least one meltable substance in at least one encasing medium, said method comprising the steps of dripping a melt of the substance into a melt of the medium, allowing the encased substance melt to solidify at least on the surface of the substance, and separating the substance from the encasing medium.

2. The method according to claim 1, wherein the melting point of the substance is at least 5° C. above the melting point of the encasing medium.

3. The method according to claim 1, wherein the melting point of the substance is between 30° and 300° C.

4. The method according to claim 1, wherein the medium is selected from the group consisting of polyethylene glycols, polypropylene glycols and mixtures thereof and the medium has melting points between 3° and 150° C.

5. The method according to claim 1, wherein the substance comprises at least one compound selected from the group consisting of oxalic acid, malonic acid, succinic acid, glutaric acid, adipic acid, pimelic acid, azelaic acid, sebacic acid, maleic acid, fumaric acid, sorbic acid, phthalic acid, terephthalic acid, citric acid, dodecanedioic acid, stearic acid, trisodium citrate, salicylic acid, glycolic acid, anhydrides of the aforementioned acids and mixtures thereof.

6. The method according to claim 1, the substance comprises at least one compound selected from the group consisting of hydrogencarbonates, alkali metal hydrogencarbonates, sodium hydrogencarbonates, potassium hydrogencarbonates, alkali metal hydrogensulfates, potassium hydrogensulfates and an eutectic mixture of potassium hydrogensulfate and sodium hydrogensulfate that comprises 60 wt % NaHSO₄ and 40 wt % KHSO₄.

7. The method according to claim 1, wherein the substance comprises a compound selected from the group consisting of phosphonates, hydroxyalkane phosphonates, aminoalkane phosphonates (1-hydroxyethane-1,1-diphosphonate (HEDP) as the disodium salt of HEDP, the tetrasodium salt of HEDP, ethylenediaminetetramethylene phosphonate (EDTMP), diethylenetriaminepentamethylene phosphonate (DTPMP), higher homologs of EDTMP, higher homologs of DTPMP, neutrally reacting sodium salts of EDTMP, neutrally reacting sodium salts of DTPMP, hexasodium salt of EDTMP, heptasodium salt of DTPMP and the octasodium salt of DTPMP.

8. The method according to claim 1, wherein the substance comprises a compound selected from the group consisting of sugars, monosaccharides, disaccharides, trisaccharides, tetrasaccharides, pentasaccharides, hexasaccharides, sucrose, a mixture of
6-0-∝-D-glucopyranosyl-D-sorbitol (I, 6-GPS) and
4-0-∝-D-glucopyranosyl-D-mannitol (I, I-GPM) and a mixture of 1,6-GPS and 1,1-GPM in which the 1,6-GPS is less than 57% of the total.

9. The method according to claim 1, wherein the substance comprises urea.

10. The method according to claim 1, wherein multiple melts of substances to be encased are dispersed simultaneously into the encasing medium.

11. The method according to claim 1, wherein the separation step is carried out by straining the encased particles out of the medium.

12. The method according to claim 11, wherein the thickness of the medium is 0.1 to 2500 μm.

13. The method according to claim 1, wherein the solidified substance has particle sizes of from 0.5 to 50 mm.

14. The method according to claim 1, wherein the substance comprises at least one compound selected from the group consisting of dodecanoic acid (lauric acid), tetradecanoic acid (myristic acid), hexadecanoic acid (palmitic acid), octadecanoic acid (stearic acid), eicosanoic acid (arachidic acid), docosanoic acid (behenic acid), tetracosanoiuc acid (lignoceric acid), hexacosanoic acid (cerotinic acid), triacontanoic acid (melissic acid), as well as the unsaturated species 9c-hexadecenoic acid (palmitoleic acid), 6c-octadeceneoic acid (petroselinic acid), 6t-octadecenoic acid (petroselaidic acid), 9c-octadecenoic acid (oleic acid), 9t-octadecenoic acid (elaidic acid), 9c, 12c-octadecadienoic acid), 9t, 125-octadecadienoic acid (linolaidic acid), and 9c, 12c, 15c-octadecatrienoic acid (linolenic acid) and mixtures thereof.

15. A method of encasing a substance selected from the group consisting of citric acid, citrates, trisodium citrate and citric acid anhydride in at least one encasing medium, said method comprising the steps of dripping a melt of the substance onto a salt of the medium, allowing the encased substance melt to solidify at least on the surface of the substance and separating the substance from the encasing medium.

16. A substance obtained by a method for encasing a substance in at least one encasing medium, said method comprising the steps of dripping a melt of the substance into a melt of the medium, allowing the encased substance melt to solidify at least on the surface of the substance and separating the substance from the medium.

17. The substance according to claim 16, wherein the substance comprises at least one compound selected from the group consisting of oxalic acid, malonic acid, succinic acid, glutaric acid, adipic acid, pimelic acid, azelaic acid, sebacic acid, maleic acid, fumaric acid, sorbic acid, phthalic acid, terephthalic acid, citric acid, dodecanedioic acid, stearic acid, trisodium citrate, salicylic acid, glycolic acid, anhydrides of the aforementioned acids and mixtures thereof.

18. The substance according to claim 16, wherein the substance comprises a compound selected from the group consisting of phosphonates, hydroxyalkane phosphonates, aminoalkane phosphonates (1-hydroxyethane-1,1-diphosphonate (HEDP) as the disodium salt of HEDP, the tetrasodium salt of HEDP, ethylenediaminetetramethylene phosphonate (EDTMP), diethylenetriaminepentamethylene phosphonate (DTPMP), higher homologs of EDTMP, higher homologs of DTPMP, neutrally reacting sodium salts of EDTMP, neutrally reacting sodium salts of DTPMP, hexasodium salt of EDTMP, heptasodium salt of DTPMP and the octasodium salt of DTPMP.

19. The substance according to claim 16, wherein the substance comprises a compound selected from the group consisting of sugars, monosaccharides, disaccharides, trisaccharides, tetrasaccharides, pentasaccharides, hexasaccharides, sucrose, a mixture of
6-0-∝-D-glucopyranosyl-D-sorbitol (I, 6-GPS) and
4-0-∝-D-glucopyranosyl-D-mannitol (I, I-GPM) and a mixture of 1,6-GPS and 1,1-GPM in which the 1,6-GPS is less than 57% of the total.

20. The substance according to claim 16, wherein the substance comprises urea.
